(19) United States
(12) Patent Application Publication
(10) Pub. No.: US 2012/0101506 A1
(43) Pub. Date: Apr. 26, 2012

(54) TISSUE RETRACTOR APPARATUS AND METHODS OF USE

(75) Inventors: John SHIM, Odessa, FL (US); Thomas PURCELL, Del Mar, CA (US); Clark HUTTON, Oceanside, CA (US)

(73) Assignee: Alphatec Spine, Inc., Carlsbad, CA (US)

(21) Appl. No.: 13/278,871

(22) Filed: Oct. 21, 2011

Related U.S. Application Data
(60) Provisional application No. 61/405,348, filed on Oct. 21, 2010.

(51) Int. Cl. A61B 17/70; A61B 17/88

(52) U.S. Cl. 606/104; 606/86 A

(57) ABSTRACT

Apparatus and methods of use for a tissue retractor having a first retractor with a first main frame with a first distal blade and a second retractor having a second main frame with a second distal blade, the first distal blade and second distal blade being configured to engage each other on opposite sides of a spinal implant creating an expandable passageway p between the first main frame and second main frame.

### CROSS-REFERENCE TO RELATED APPLICATIONS

This application claims priority to U.S. Provisional Patent Application No. 61/405,348, filed Oct. 21, 2010, which is incorporated herein by reference.

### FIELD

The invention generally relates to minimally invasive spinal surgery, and more specifically, a tissue retraction device.

### BACKGROUND

Traditional surgical procedures for spinal surgeries, as opposed to minimally invasive surgeries, can cause significant trauma in intervening tissues and thus often require a long incision and prolonged retraction of tissues, which may lead to a post-surgical recovery time of several weeks. With the development of minimal invasive surgical techniques to spinal applications, the risk of damaging intervening tissues, the degree of post-surgical pain and the amount of recovery time are significantly reduced.

With the development of minimal invasive spinal surgeries, further improvements in devices for such procedures remain crucial. Among those devices, a minimally invasive retractor simpler than traditional minimally invasive retractors is needed.

In a minimally invasive spinal surgery, a retractor is a device by which a surgeon can either actively separate the edges of a surgical incision or wound, or can hold back underlying organs and tissues, so that body parts under the incision may be accessed. Current tissue retractors include many working parts that can malfunction and include complex technical aspects. The present invention attempts to solve these problems as well as others.

### SUMMARY

Provided herein are methods, systems, and apparatuses for a tissue retractor. The tissue retractor having a first retractor with a first main frame with a first distal blade and a second retractor having a second main frame with a second distal blade, the first distal blade and second distal blade being configured to engage each other on opposite sides of a spinal implant creating an expandable passageway p between the first main frame and second main frame. In some embodiments, each the main frame includes a proximal section, a middle section, and a distal section; the distal sections of the first retractor and the second retractor include the first distal blade and the second distal blade, respectively. In some embodiments, the first blade includes a first hook section and the second blade includes a second hook section; the first blade operably couples with the second blade to clamp down on a medical device, a spinal implant, a medical screw, or the like, while allowing a surgeon to hold back soft tissue with the first blade and the second blade.

The methods, systems, and apparatuses are set forth in part in the description which follows, and in part will be obvious from the description, or can be learned by practice of the methods, systems, and apparatuses. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, systems, and apparatuses, as claimed.

### BRIEF DESCRIPTION OF THE DRAWINGS

In the accompanying figures, like elements are identified by like reference numerals among the several preferred embodiments of the present invention.

FIG. 1 is a side perspective view of the tissue retractor including the first retractor and second retractor.

FIG. 2A is a side view of one embodiment of the interior face of the first retractor with the perforated frame shown in phantom; FIG. 2B is a side view of one embodiment of the exterior face of the first retractor with the perforated frame shown in phantom; FIG. 2C is a side view of the interior face of the first retractor in a left-handed form; and FIG. 2D is a side views of the interior face of the first retractor in a right-handed form.

FIG. 3A is a side view of one embodiment of the first retractor; FIG. 3B is zoomed side view of one embodiment of the junction between the perforated frame and the first blade; FIG. 3C is a zoomed side view of one embodiment of the proximal end of the first retractor and the curved section; and FIG. 3D is a zoomed side view of one embodiment of the distal end of the first retractor and the first blade and the first clamp section.

FIG. 4A is a zoomed front view of one embodiment of the front face of the clamp section; and FIG. 4B is a zoomed side front view of one embodiment of the front face of the first retractor and the second retractor.

FIG. 5A is a perspective view of one embodiment of the pedicle screw for the use in surgery and medical devices; and FIG. 5B is a perspective view of one embodiment of the pedicle screw.

FIG. 6A is a side view of one embodiment of the first retractor and the second retractor operably coupled with the pedicle screw; and FIG. 6B is a zoomed side view of one embodiment of the first retractor and the second retractor operably coupled with the pedicle screw.

FIG. 7 a perspective view of one embodiment of the first retractor operably coupled with the pedicle screw.

FIG. 8 is a schematic diagram illustrating a flow chart for using the tissue retractor in accordance with one embodiment.

FIG. 9 is a gelpi instrument that may be used in one embodiment for displacing the first retractor and the second retractor along the z-axis, either towards the interior face or exterior face of the first and second retractor.

### DETAILED DESCRIPTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

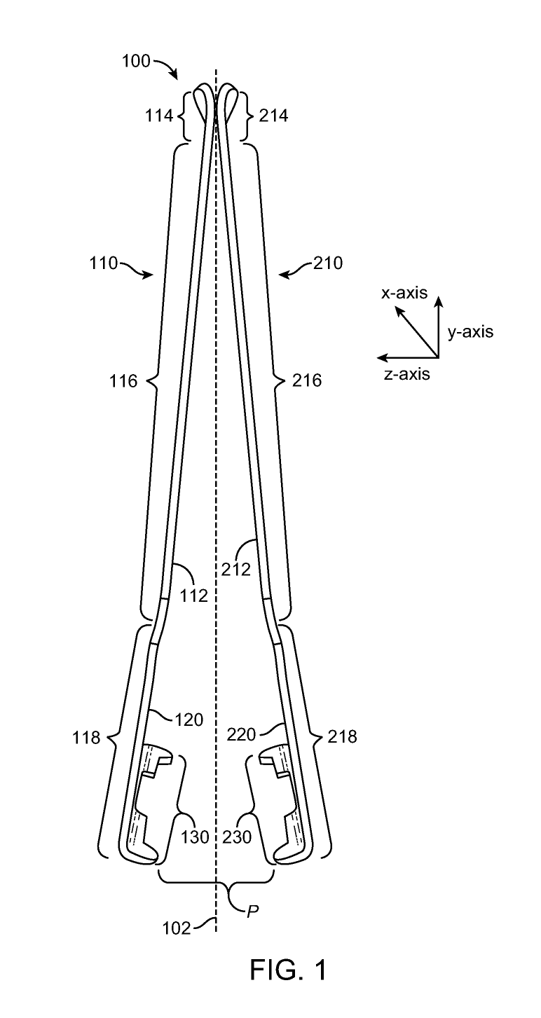

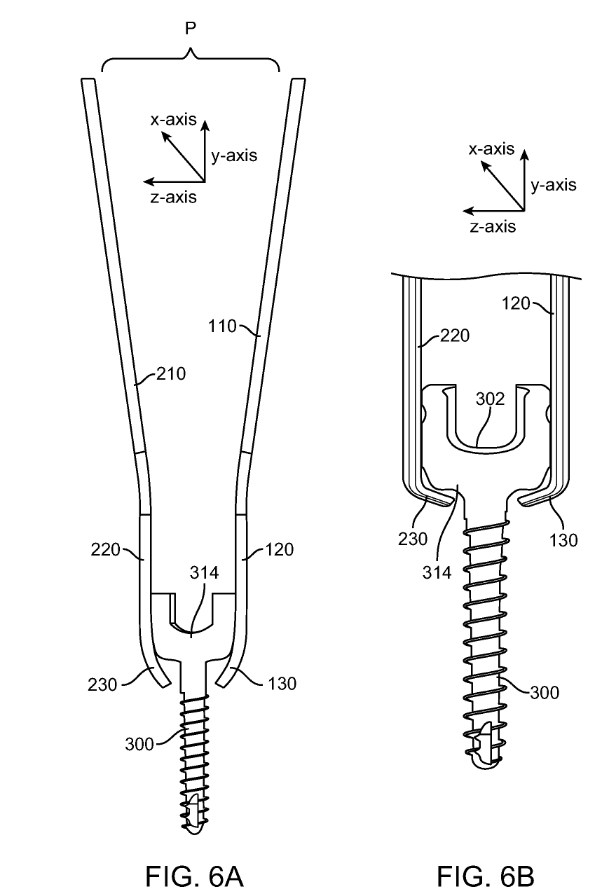

Generally speaking, the tissue retractor 100 comprises a first retractor 110 and a second retractor 210, as shown in FIG. 1. The first retractor 110 and the second retractor 210 are substantially similar in shape, structure, and function, whereby the first retractor 110 is reciprocal to the second retractor 210 to provide a clamping force between the first retractor 110 and the second retractor 210 and expose or retract bodily tissue and provide a passageway P therebetween. FIG. 6A shows the open passageway P when the first and second retractors engage the implant are retracted at the top. Passageway P may be within any opening, puncture, wound, or incision through the tissue to a body cavity; hence, passageway P may comprise an incision, other opening formed by the cutting or removal of tissue, bone, or cartilage, a percutaneous opening through tissue, or the like. The longitudinal axis 102 of the first and second retractors 110 and 210 is generally shown in the y-axis direction, while the transverse axis is generally shown in the x-axis. The first retractor 110 includes a main frame 112, which includes a proximal section 114, a middle section 116, and a distal section 118. The second retractor 210 includes a main frame 212, which includes a proximal section 214, a middle section 216, and a distal section 218. The distal sections 118 and 218 include a first blade 120 and a second blade 220, respectively. The first blade 120 includes a first hook section 130 and the second blade 220 includes a second hook section 230. The first blade 120 operably couples with the second blade 220 to clamp down on a medical device, a spinal implant, a medical screw, or the like, while allowing a surgeon to hold back or retract soft tissue with the first blade 120 and the second blade 220 and move in and out any surgical tools in passageway P between the first and second retractor to perform a surgical procedure

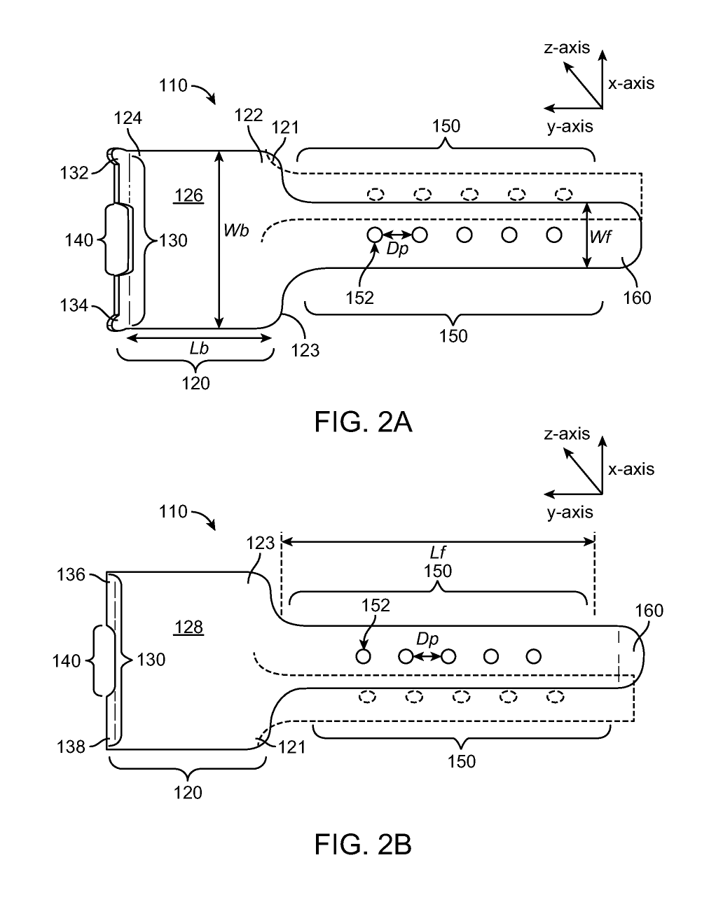

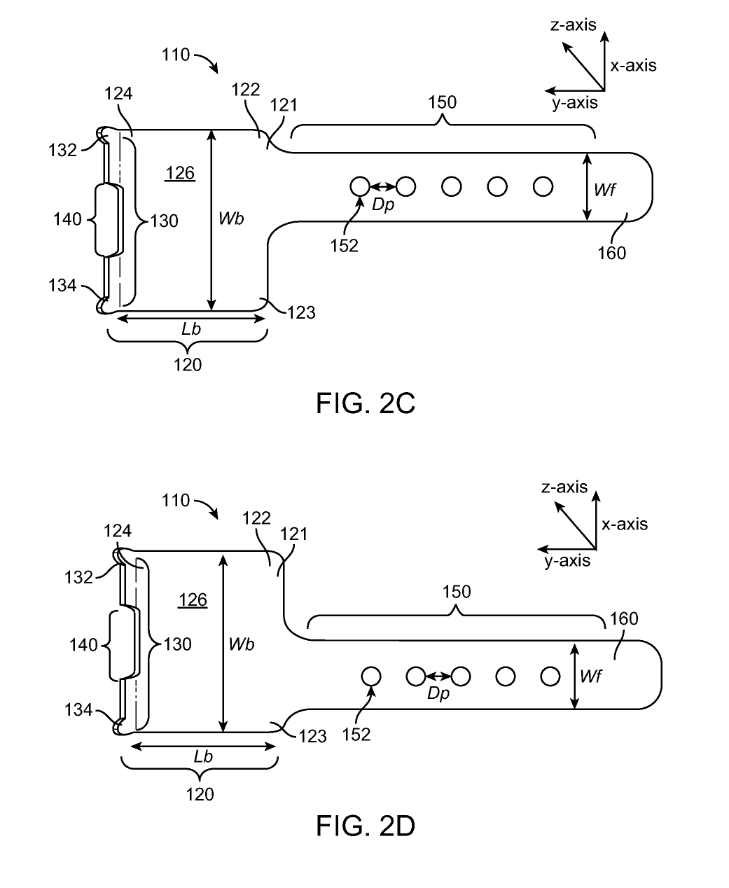

As shown in FIGS. 2A-B, the first retractor 110 includes a central axis extending along the longitudinal axis or y-direction. The first blade 120 includes a proximal portion 122, a distal portion 124, an interior face 126, and an exterior face 128. The distal portion 124 of the first blade 120 includes the first hook section 130. The first hook section 130 includes a first clamp 132, a second clamp 134 and a center cuff 140, whereby the first clamp 132 and the second clamp 134 project towards the interior face 126 along the z-axis of the first blade 120. The proximal portion 122 of the first blade 120 includes a curved design towards the central axis of the first retractor 110, which connects the first blade 120 to the middle section 116. The middle section 116 includes a perforated frame 150 including a plurality of holes 152 in a spaced apart relationship along the longitudinal axis of the first blade 120, whereby the holes 152 are separated by a diameter Dp. The perforated frame 150 is generally shown to longitudinally extend from the middle portion of the first blade 120, such that the first retractor 110 is generally symmetrical along the longitudinal axis. Alternatively, the perforated frame 150 may extend from a top portion 121 or a bottom portion 123 of the first blade 120, such that the first retractor is generally asymmetrical along the longitudinal axis, as shown in phantom in FIGS. 2A-2B. An asymmetrical perforated frame 150 allows for generally right-handed or left-handed asymmetrical retractors 110 and 210 to allow a surgeon or operator to curve the retractors 110 and 210 into an incision site and extend the retractors either upward or downward from the original placement or incision site. The interior face 126 is shown to be a flat surface in one embodiment, although, the interior face 126 may include a curved or angled surface such as to conform to the surface profile of a medical device or a pedicle screw.

As shown in FIG. 2A, the first blade 120 includes a width Wb and a length Lb, where the width Wb is generally shown along the x-axis direction of the first retractor 110 and the length Lb is generally shown along the y-axis of the first retractor 110. The dimensions for Wb and Lb may be within any particular parameters or usage that should be noticed for the first blade, which may either be selected in terms of retraction abilities or clamping force on a medical device. In one embodiment, the width Wb and the length Lb are generally selected for the type of bodily tissue to be retracted or to be exposed. Alternatively, the width Wb of the first blade 120 may be equal, greater than, or less than the length Lb. The perforated frame 150 also includes a width Wf, whereby the blade width Wb is greater than the perforated frame width Wf. In one embodiment, the holes 152 are centrally located in the perforated frame 150, which allow for a “gelpi” like surgical instrument to push the first retractor 110 apart from the second retractor 210 to provide the passageway P in between the first and second retractor. A gelpi instrument 400 is shown in FIG. 9, and explained further below.

As shown in FIGS. 2A-B, the proximal portion 114 of the first retractor 110 includes a curved portion 160, which curves towards the exterior face 128 of the first retractor 110 generally in the z-axis direction. The curved portion 160 allows for a surgeon or operator a handle or ability to retract bodily tissue and/or clamp down on a medical device. As shown in FIG. 2B, the first hook section 130 includes a first curved element 136 and a second curved element 138, which curves towards the interior face 126 of the first blade 120 to connect the exterior face 128 with the first clamp 132 and second clamp 134, respectively. The first and second curved elements 136 and 138 may be curved at any particular angle αt which may be suitable for retracting tissue or penetrating a tissue opening or incision. The general shape of the first blade 120 and the second blade 220 may be polygonal, rectangular, square, trapezoidal, elliptical, triangular, parabolic, C-shaped, U-shaped, and the like. The general shape of the perforated frame 150 may be polygonal, rectangular, square, trapezoidal and the like, or selected as to advance the first blade 120 through a tissue opening or incision. The perforated frame 150 includes a length Lf, which may be selected as to advance the first blade 120 through a tissue opening or incision. Generally, the length Lf is greater than the length Lb of the first blade 120. The exterior surface 128 is generally shown to be a flat surface in one embodiment; however, alternatively, the exterior surface 128 may be a curved or angled surface to provide for alternative retracting techniques.

FIG. 2C is a side view of the interior face of the first retractor in a left-handed form; and FIG. 2D is a side views of the interior face of the first retractor in a right-handed form. In the left-handed form of the first retractor 110, the perforated frame 150 longitudinally extends from the top portion 121 of the first blade 120, while the second retractor 210 would include the perforated frame 150 longitudinally extending from the bottom portion of the second blade 220. In the right-handed form of the first retractor 110, the perforated frame 150 longitudinally extends from the bottom portion 123 of the first blade 120, while the second retractor 210 would include the perforated frame 150 longitudinally extending from the top portion of the second blade 220. The right-handed or left-handed asymmetrical retractors 110 and 210 allow a surgeon or operator to curve the retractors 110 and 210 into an incision site and extend the retractors either upward or downward from the original placement or incision site.

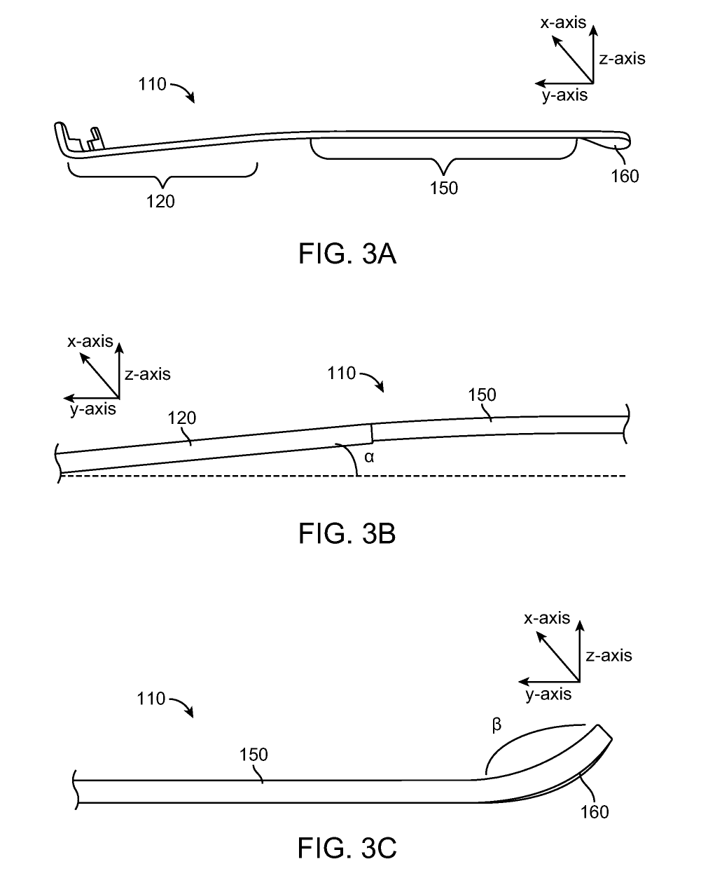

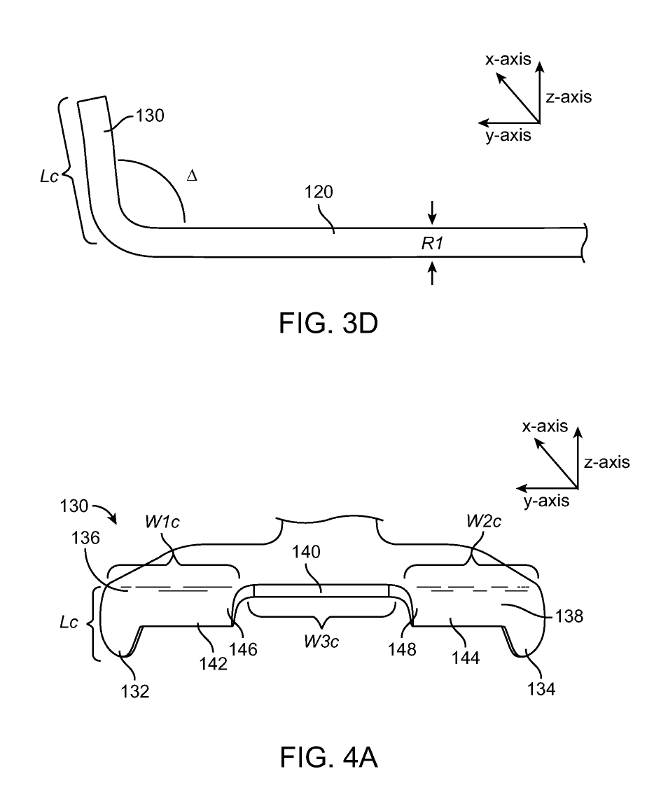

As shown in FIG. 3A, the first blade 120 is offset at a first angle with respect to the perforated frame 150 along the y-axis and the curved portion 160 is offset at a second angle with respect to the perforated frame 150 along the y-axis, when the perforated frame 150 is substantially parallel to the y-axis of the first retractor 110. The offset angles for the first and second retractor are set to allow retraction of bodily tissue while being able to hold or clamp down on a medical device and provide the passageway P. As shown in FIG. 3B, the first blade 120 is offset at an angle α with respect to the perforated frame 150 and along the longitudinal axis of the first retractor 110. The angle α may be within a range of angles to assist with the clamping of the pedicle screw or spinal implant such as to be displaced away from bodily tissue that is retracted. In one embodiment, the angle α may be between about 0 and 45 degrees, alternatively, between about 10 and 35 degrees, alternatively, between about 15 and 25 degrees, alternatively, the angle α may be selected to optimize retraction of bodily tissue and/or providing a clamping force upon a medical device and/or providing for passageway P between the first and second retractor.

As shown in FIG. 3C, the curved portion 160 is offset at an angle β with respect to the perforated frame and along the longitudinal axis of the first retractor 110. The angle β may be within the range or selected as to assist the surgeon pushing the first and second retractor into an incision point and/or with displacing the first retractor from the second retractor to provide for passageway P. In one embodiment, the angle β may be between about 0 and 45 degrees, alternatively, between about 10 and 35 degrees, alternatively, between about 15 and 25 degrees.

As shown in FIG. 3D, the first clamp section 130 is offset at an angle Δ with respect to the longitudinal axis of the first blade 120, when the longitudinal axis of the first blade 120 is substantially parallel with the y-axis of the first retractor 110. The angle Δ may be within the range as to assist the surgeon pushing the first and second retractor into an incision point or with clamping the first retractor and the second retractor together. The angle Δ may be selected or within the range as to provide for the clamping force upon a medical device and/or penetrating an incision point or bodily tissue. In one embodiment, the angle Δ may be between about 45 and 170 degrees, alternatively, between about 65 and 150 degrees, alternatively, between about 85 and 125 degrees.

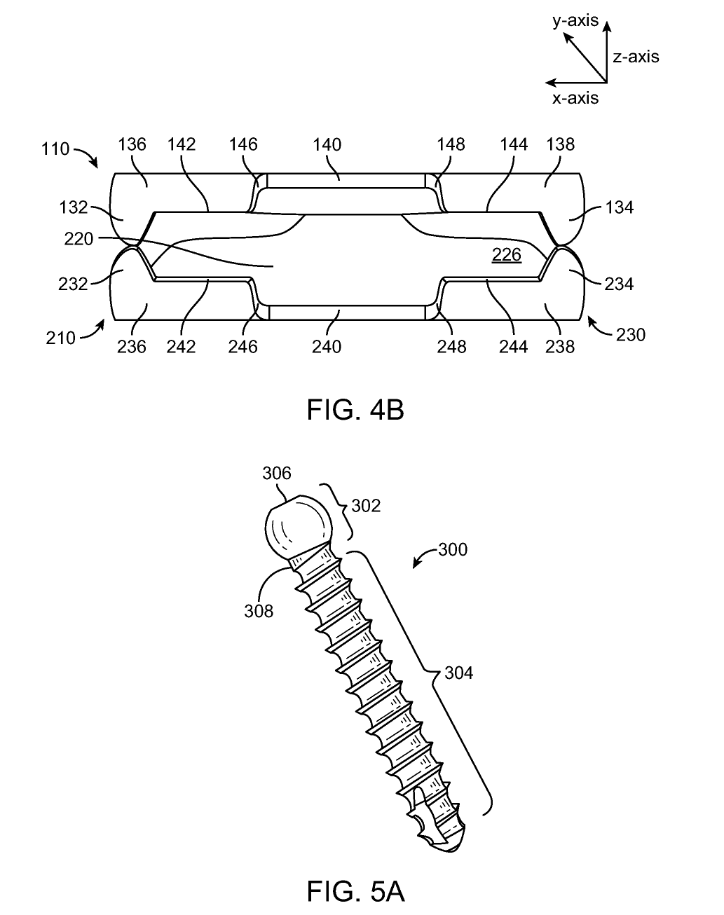

The first blade 120 and the first retractor 110 include a thickness R1, which may be maintained throughout the entire longitudinal length of the first retractor 110. In one embodiment, the thickness R1 is minimal to allow for minimally invasive surgery. Alternatively, the thickness R1 is varied throughout the length of the first and second retractor or selected for a particular retraction of bodily tissue or incision point. The first clamp section 130 includes a length Lc, which is the length of the first clamp 132 and the second clamp 134, as shown in FIG. 4A. The length Lc of the first clamp and second clamp 132 and 134 may be selected for any particular medical device that is to be clamped and maintained between the first and second clamp 132 and 134. For example, the length Lc may be longer if the medical device includes a thicker profile, alternatively, the length Lc may be shorter in length if the medical device includes a smaller or thinner profile.

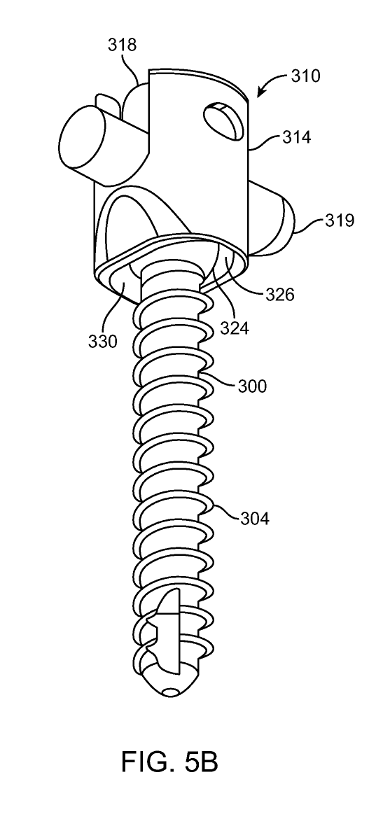

As shown in FIG. 4A, the first clamp section 130 includes a central cuff 140 in the middle portion of the first clamp section 130, where the central cuff 140 is bordered and positioned between the first curved element 136 and the second curved element 138. The first clamp 132 and the second clamp 134 project generally towards the interior face, away from the first and second curved elements 132 and 134, and along the z-axis at the angle Δ of the first clamp section 130. The first clamp 132 includes a first lip 142 that generally connects with the first curved element 136 and the central cuff 140. The second clamp 134 includes a second lip 144 that generally connects with the second curved element 138 and the central cuff 140. The first lip 142 includes a first rim 146 and the second lip 144 includes a second rim 148, such that a head portion of a medical device may fit within the central cuff 140. The first curved element 136 includes a width W/c, the second curved element 138 includes a width W2c, and the central cuff 140 includes a width W3c. In one embodiment, the width W3c is shaped or altered to fit the dimensions of a medical device, spinal implant, or pedicle screw. The diameters W1c, W2c, and W3c make up the width Wb of the first blade 120. The diameters W1c, W2c, and W3c may be adjusted as to fit the width and width dimensions of a particular medical device. The first curved element 136 and the second curved element 138 operably couple with the central cuff 140 to maintain or hold a medical device, spinal implant, or pedicle screw, as shown in FIGS. 5A and 5B.

As shown in FIG. 5A, a pedicle screw body 300 has a screw (polyaxial or otherwise) design for the purpose of attaching the spine. Pedicle screw body 300 typically includes a head portion 302, a threaded shaft portion 304, a top unthreaded portion 308, and an engagement surface 306 in the head portion 302 for use in driving the screw 300 into vertebrae (not shown). The head portion 302 may be spherical, rounded, hexagonal, polygonal, rectangular, square, and the liked shapes for surgical screws.

A pedicle screw assembly 310 is shown in FIG. 5B comprising the pedicle screw 300, a body member 314, a bushing 316 and a locking cap 318. The pedicle screw assembly 310 is used with at least one other such assembly and a stabilization or fixation rod 319 to connect the assemblies and stabilize the vertebras into which the assemblies are inserted. The pedicle screw 300 preferably employed in assembly 310 has a spherical head 302 defining a slot or recess 306 therein used to drive the screw into the bone. The rounded surface 324 defined by the lower portion of screw head 302 rests upon and mates with a rounded interior surface 326 formed in the inner or lower end of the body member 314 of the assembly 310 so as to form a modified ball joint that provides the desired variable angular movement of the body member with respect to the embedded pedicle screw. The threaded shaft portion 304 of screw 300 extends therefrom through the opening 330 in the lower end of body member 314.

As shown in FIG. 4B, the central cuff 140 is shaped as to fit the top head portion 302 of the pedicle screw 300 or the body member 314 of the pedicle screw assembly 310. The first lip 142 includes the first rim 146 and the second lip 144 includes the second rim 148, such that the top head portion 302 of the pedicle screw is secured to the first blade 120 and the first retractor 110. The first clamp 132 and the second clamp 134 ensure that if the pedicle screw falls out of the first rim 146 and the second rim 148, the pedicle screw is still secured within the first blade 120 and the first clamp section 130.

As shown in FIG. 4B, the first retractor 110 is shown on top with the second retractor 210 on the bottom, when the first retractor 110 is operably coupled with the second retractor 210. For clarity purposes, the second retractor 210 includes the second blade 220 and a second hook section 230. The second hook section 230 includes a first clamp 232, a second clamp 234 and a center cuff 240, whereby the first clamp 232 and the second clamp 234 project towards the interior face 226 along the z-axis of the second blade 220, and the center cuff 240 is positioned between the first and second clamps 232 and 234. The second hook section 230 includes a first curved element 236 and a second curved element 238 on opposing ends of the second hook section 230, which curves towards the interior face 226 of the second blade 220 to connect the exterior face with the first clamp 232 and second clamp 236, respectively. The first clamp 232 includes a first lip 242 that generally connects with the first curved element 236 and the central cuff 240. The second clamp 234 includes a second lip 244 that generally connects with the second curved element 238 and the central cuff 240. The first lip 242 includes a first rim 246 and the second lip 244 includes a second rim 248 to connect to the center cuff 240, such that the top head portion 302 of the pedicle screw is secured in between the second retractor 210 and the first retractor 110 when the head portion is placed between the center cuffs 140 and 240.

As shown in FIGS. 6A and 6B, the first retractor 110 and second retractor 210 engage with the head portion 302 or body member 314 of the pedicle screw assembly 310 through a tissue opening or incision (not shown) and are separated creating passageway P. The top head portion of the screw rests on top of the first and second blades 120 and 220 and engaged with the first and second clamp sections 130 and 230. As can be seen in FIG. 6B, the first clamp sections 130 230 slip under the pedicle screw 300 and prevent the first retractor 110 and second retractor 210 from moving in the longitudinal direction, generally shown in the y-axis. In one embodiment, the pedicle screw 300 may include a screw lip 310, which is engaged and secured by the first and second lips of the first and second clamp section 130 and 230.

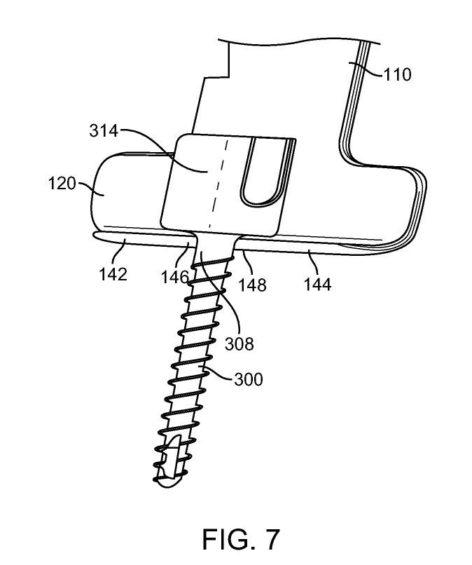

In one embodiment, as shown in FIG. 7, the pedicle screw 300 is positioned between the first rim 146 and the second rim 148 of the first retractor 110, which engage and secure the pedicle screw 300 at the top unthreaded portion 308 that is seated in the central cuff 140. The body member 314 seats and abuts the interior face 126 of the first blade 120, whereby the first rim 146 and the second rim 148 operably fit to the top unthreaded portion 308 of the shaft 304.

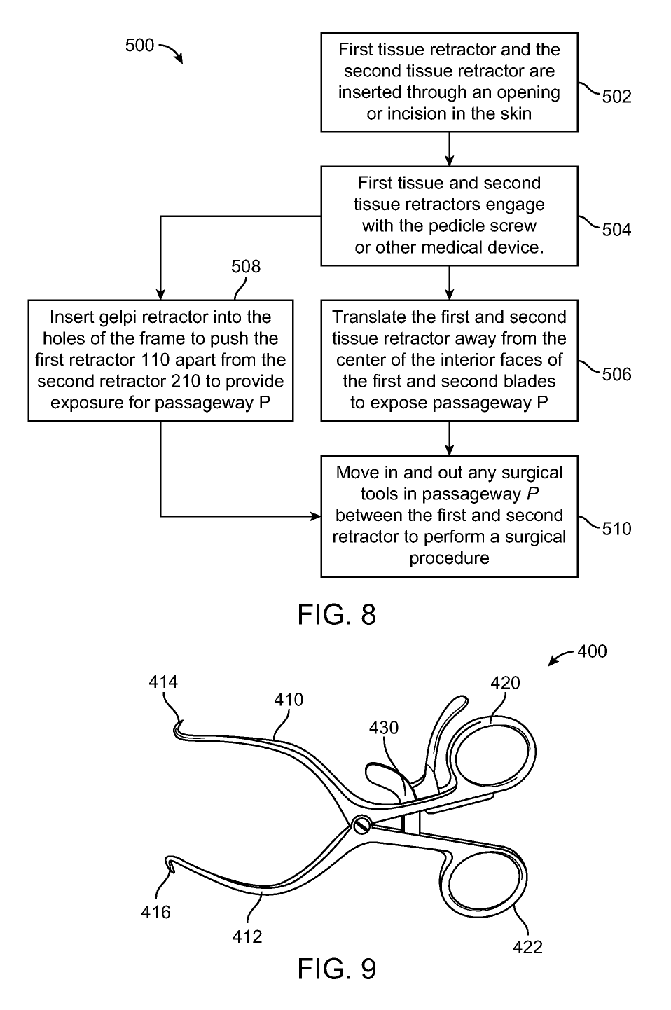

FIG. 8 illustrates a method 500 for retracting body tissue using the tissue retractor 100. In step 502, the first tissue retractor and the second tissue retractor are inserted through a tissue opening or incision in the skin proximate a pedicle screw or other medical device. In step 504, the first and second retractors engage with the pedicle screw or other medical device at a location where bodily tissue needs to be retracted. The locations for tissue retraction can be any location on or within the body, such as a location where a surgical procedure is being or will be performed. In one embodiment, the tissue retractor can be used to expose spinal structures during spinal surgery, which allows for minimal disruption of spinal muscles and sensitive elements of the posterior, lateral, and anterior regions of the spine. The tissue retractor 100 can also be used in the thoracolumbar region, as well as, sacral and cervical regions of the spine, or any other vertebrae regions.

In step 506, the operator translates the first and second tissue retractor away from the center of the interior faces of the first and second blades and expose passageway P, whereby the exterior faces of the first and second tissue retractors retract the bodily tissue. The first and second tissue retractors may be translated the same distance away from the interior faces or different distances away. By forcing the first and second retractors apart, the engaged bodily tissue is also spread/forced apart and expose passageway P, as illustrated in step 506. And by spreading the tissue apart, in step 510, the surgeon or other medical professional can place the medical device or pedicle screw at a surgical location and easily move in and out any surgical tools in between the first and second retractor through passageway P, such as a hexagonal driver or threaded cannula to screw in the pedicle screw, or perform any other surgical procedure.

Alternatively, in Step 508, the gelpi instrument 400 may be inserted into the holes 152 in the perforated frame 150 to push the first retractor 110 apart from the second retractor 210 and provide the passageway P in between the first and second retractor. As shown in FIG. 9, the gelpi instrument 400 is a self-retaining, small spreader suitable for small sites that includes two blades 410 and 412, hinged in the middle, separate as the handles 420 and 422 are closed and are held open by a ratchet 430. The blades turn down at right angles at their tips 414 and 416, and are bowed towards each other so that they push the edges of the incision apart and retain themselves in it.

The proposed tissue retractor approach is simpler than the traditional Minimally Invasive Surgery (“MIS”) retractor approach and may remove the somewhat technically challenging aspects of using a traditional minimally invasive retractor. These include small mechanism to operate, metallic frame blocking x-ray, breakage of retractor mechanism, and so on. For instance, conventional MIS retractors developed before the modified Comis version, have been created with extra long handles to keep the assistants' hands out of the surgeon's way, and out of the field of exposure. However, these long handles impart greater forces on the soft tissues and cause more soft tissue damage. The thin, simple design of the proposed soft-tissue reactor may produce the same soft tissue retraction with the MIS and other retractors with complex mechanisms.

The proposed soft tissue retractor for lumbar spine surgery is thin, structural blade with distal and proximal features. These features are designed to work with the anatomy, the spinal implant, and other related surgical tools to deliver spinal implants during surgery. These retractors are designed to work as a pair. The current design has small features at the distal tip to hook under the spinal implant.

As can be understood by one skilled in the art, the retractor 100 and/or any of its components may have any size, shape, length, thickness, height, weight, or any other parameters. Such parameters may be selected by the surgeon (or other qualified professional) for performance of specific procedures. Further, the retractor 100 and/or any of its components may be manufactured from metal, plastic, synthetic material, or other suitable materials, or any combination thereof.

In some embodiments, the blades 110, 210 are included in various lengths and configurations, and may also include various features to accommodate different applications for the retractor. The blades 110, 210 can be used to form a closed exposure to the surgical site and can be installed easily while at this position. The blades can be constructed of various materials to aid in radio translucency, strength, flexibility, and integration with anatomy etc.

Even though the illustrated embodiment of the surgical retractor includes two retractors (or sections), the present invention is not limited to having two retractors. As can be understood by one skilled in the art, there can be any number of retractors (or sections) that are configured to retract tissue away from the surgical site.

The proposed new minimally invasive reactor may allow surgeons to create a small opening in the lumbar muscles and benefit from similar results compared to traditional Minimally Invasive Retractors, (MIS) retractors. MIS retractors protrudes as little as 25 mm above the skin level during surgery, provides full circumferential view of the acetabulum, incorporates soft-tissue friendly curved blades, and allows controllable forces imparted on soft tissue during retraction.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

The invention claimed is:

1. A tissue retractor comprising:
a first retractor having a first main frame with a first distal blade; and
a second retractor having a second main frame with a second distal blade;
the first distal blade and second distal blade being configured to engage each other on opposite sides of a spinal implant creating an expandable passageway P between the first main frame and second main frame.

2. The tissue retractor of claim 1, wherein the first distal blade includes a first hook section and the second distal blade includes a second hook section.

3. The tissue retractor of claim 2, wherein the first distal blade and second distal blade include an interior face and an exterior face, the first hook section and the second hook section include a first clamp, a second clamp, and a center cuff, whereby the first clamp and the second clamp project towards the interior face of the first distal blade and second distal blade.

4. The tissue retractor of claim 1, wherein the first and second main frames each include a proximal section, a middle section, and a distal section, the first and second distal blades being on distal section.

5. The tissue retractor of claim 4, wherein the proximal sections of the first retractor and the second retractor include a curved portion that curves towards an exterior portion of the first retractor and the second retractor.

6. The tissue retractor of claim 1, wherein the first and second main frames include a plurality of holes configured to engage an instrument to translate the first retractor apart from the second retractor to expand the passageway P.

7. The tissue retractor of claim 1, wherein the first and second main frames include a frame width Wf and the first and second distal blades include a blade width Wb, whereby the blade width Wb is greater than the frame width Wf.

8. The tissue retractor of claim 1, wherein the first and second distal blades are symmetrical with first and second main frames with respect to a y-axis.

9. The tissue retractor of claim 1, wherein the first and second distal blades are asymmetrical with first and second main frames with respect to a y-axis.

10. A tissue retractor comprising:
a first retractor and a second retractor, whereby the first retractor and the second retractor are configured to provide a clamping force between the first retractor and the second retractor to expose bodily tissue and a passageway P;
the first retractor and the second retractor each include a main frame, wherein the main frame includes a proximal section, a middle section, and a distal section;
the distal sections of the first retractor and the second retractor include a first blade and a second blade, respectively, wherein the first blade includes a first hook section and the second blade includes a second hook section;
the first blade operably couples with the second blade to clamp down on a medical device, a spinal implant, a medical screw, or the like, while allowing a surgeon to hold back soft tissue with the first blade and the second blade.

11. The tissue retractor of claim 10, wherein the first blade and second blade include a proximal portion, a distal portion, an interior face, and an exterior face;
the distal portion of the first blade and second blade includes the first hook section and second hook section, respectively;
the first hook section and the second hook section include a first clamp, a second clamp, and a center cuff, whereby the first clamp and the second clamp project towards the interior face along a z-axis of the first blade and second blade;
the proximal portion of the first blade and the second blade includes a curved design towards the central axis connecting the first blade to the middle section.

12. The tissue retractor of claim 10, wherein the middle section of the first retractor and the second retractor include a perforated frame including a plurality of holes in a spaced apart relationship along the longitudinal axis of the first retractor and the second retractor, whereby the holes are separated by a diameter Dp.

13. The tissue retractor of claim 12, wherein the first blade and the second blade include a width Wb and a length Lb, the width Wb is generally shown along an x-axis direction and the length Lb is generally shown along a y-axis.

14. The tissue retractor of claim 13, wherein the width Wb and the length Lb are generally selected for the type of bodily tissue to be retracted or to be exposed.

15. The tissue retractor of claim 13, wherein the perforated frame includes a width Wf, whereby the blade width Wb is greater than the perforated frame width Wf.

16. The tissue retractor of claim 11, wherein the proximal portion of the first retractor and the second retractor include a curved portion that curves towards the exterior portion of the first retractor and the second retractor generally in the z-axis direction.

17. The tissue retractor of claim 11, wherein the first hook section and the second hook section includes a first curved element and a second curved element that curves towards the interior face of the first blade and the second blade, respectively, to connect the exterior face with the first clamp and second clamp.

18. The tissue retractor of claim 10, wherein the first blade and the second blade include a shape selected from the group consisting of polygonal, rectangular, square, trapezoidal, elliptical, triangular, parabolic, C-shaped, U-shaped, and the like.

19. A method if using a tissue retractor in a surgical procedure comprising:
inserting a first retractor and a second retractor of the tissue retractor through an opening or incision in the skin;
engaging the distal ends of the first and second retractors on opposite sides of a spinal implant;
translating the proximal ends of the first and second retractors away from each other forming a passageway P; and
moving one or more surgical tools through passageway P to perform the surgical procedure.

20. The method of claim 19, wherein:
the first retractor having a first main frame with a first distal blade; and
the second retractor having a second main frame with a second distal blade;
the first distal blade and second distal blade being configured to engage each other on opposite sides of the spinal implant creating the expandable passageway P between the first main frame and second main frame.
